(19) United States
(12) Patent Application Publication
(10) Pub. No.: US 2004/0067547 A1
(43) Pub. Date: Apr. 8, 2004

(54) Rapid method for detecting micro-organisms and evaluating antimicrobial activity

(76) Inventors: Stuart Harbron, Herts (GB); Gordon Chalmers Mellis Young, Essex (GB); Phillip Brown, Clacton on Sea (GB)

Correspondence Address: Bradley N. Ruben, PC, Suite 5A, 463 First St., Hoboken, NJ, 07030-1859, US

(21) Appl. No.: 10/454,563

(22) Filed: Jun. 4, 2003

Related U.S. Application Data
(63) Continuation of application No. 09/647,572, filed on Apr. 2, 2001, now abandoned.
Application No. 09/647,572 is a 371 of international application No. PCT/GB99/01001, filed on Mar. 30, 1999.

(30) Foreign Application Priority Data
Mar. 31, 1998 (GB) 9806759.8
Sep. 24, 1998 (GB) 9820703.8
Nov. 19, 1998 (GB) 9825222.4

(51) Int. Cl. C12Q 1/04

(52) U.S. Cl. 435/34

(57) ABSTRACT

A method of identifying one or more micro-organisms in a fluid comprising the steps of:—

(i) taking a sample of the fluid containing a representative sample of any micro-organism(s) present;

(ii) optionally culturing the sample if necessary to increase the number of micro-organisms for a pre-determined range;

(iii) measuring the zeta potentials of any micro-organisms present;

(iv) optionally normalising the measurements taken in step (iii) such that they relate to standard conditions;

(v) comparing said measured zeta potentials with a table of the zeta potentials of known micro-organisms to determine which, if any, of the known micro-organisms are present in the fluid.

### FIELD OF THE INVENTION

The present invention relates to methods and apparatus for the detection of the presence of specific micro-organisms in a fluid. It is particularly applicable, but in no way limited, to identifying microbial pathogens in liquid solution. The invention also relates to methods for antibiotic sensitivity testing, and in particular methods for assessing the effectiveness of antimicrobial agents, for screening compounds for antimicrobial activity, and for determining an appropriate means of treatment for an unknown microbial infection.

### BACKGROUND TO THE INVENTION

There are many applications in which it is important to be able to detect the presence of a specific micro-organism. For example, in combating viral or bacterial infections, it is necessary to be able to identify the micro-organism responsible. Also in the brewing industry, it is important to be able to determine if a yeast is present in a solution and more particularly to determine its vitality and viability. In this context the term micro-organism has a broad meaning. It encompasses bacteria, viruses and fungi.

In both these examples speed of analysis is extremely important. For instance, in diagnosis of a medical problem the medical practitioner needs to know what organism is causing the symptoms within hours rather than days. The most appropriate treatment can then be started straight away, giving the patient the best chance of a speedy recovery. In some cases, such as meningitis, rapid and accurate diagnosis is a matter of life and death.

Under present arrangements samples are usually sent to a pathology laboratory for culture and subsequent identification. By the very nature of the procedure this takes days rather than hours. There may be more than one organism present which requires a number of different cultures in different media.

It is also known to detect microorganisms by the use of specific probes that are designed to attach themselves to the micro-organism by covalent bonding and thus to attach a marker to them so that they may be detected by some physical property. Unlike the present invention such techniques are slow and can only search for one micro-organism at a time.

The use of microelectro-kinetics to study yeast vitality and viability has been described by P. Brown in Brewer's Guardian, November 1997, pages 39-40. However, yeasts are relatively large and particularly uniform in size. Even so, it proves difficult to distinguish between commercial yeasts, such as S. cerevisiae and S. carlsbergensis, and wild yeast. Such a result mitigates against being able to differentiate between much smaller organisms such as bacteria which are much more variable in size and shape. In fact, zeta potentials of living organisms are poorly understood. The method is generally considered tedious, difficult and temperamental. Furthermore, it is not readily amenable to multiple sampling.

Similarly, when treating bacterial infections, it is important to determine whether or not the causative micro-organism is susceptible to the chosen drug or antibiotic. However, present techniques for assessing the efficacy of a drug or antibiotic against a particular micro-organism can take several days to carry out. Current practice is to prescribe a general or best guess antibiotic to combat the symptoms of the infection. In other words, therapy of the infection begins before laboratory results are available, and antibiotic sensitivity testing provides only a supplementary role in confirming that the organism is susceptible to the current agent that is being used. Usually the laboratory report will influence treatment only if the patient is failing to respond. Sometimes it may allow the clinician to change from a toxic to a less toxic agent. Antibiotic sensitivity testing may be defined as a method for determining the sensitivity of a micro-organism to a potential drug treatment. The micro-organism may be a bacterium, a fungus, a yeast, a parasite or a virus.

Currently the antibiotic sensitivity of bacteria can be assessed in a variety of ways:

1. Agar Diffusion tests, in which the antibiotic is allowed to diffuse from a point source, commonly in the form of a filter paper disc, into an agar medium which has been seeded with a test organism.

2. Broth dilution tests, in which serial dilutions of antibiotic in a suitable fluid medium are inoculated with the test organism. The highest dilution of the antibiotic to inhibit growth after overnight incubation is the minimum inhibitory concentration (MIC).

3. Agar incorporation tests, which are essentially similar to broth dilution tests except that the antibiotic dilutions are incorporated in an agar medium in a series of Petri dishes. These are spot-inoculated with a number of test organisms, usually be means of a semi-automatic inoculation device.

These methods, none of which are rapid, are described in detail in Antimicrobial Chemotherapy, edited by David Greenwood, published by Bailliere Tindall, London, pp 73-82, and incorporated herein by reference in its entirely

Newer methods of assessing bacterial susceptibility to antibiotics are being developed: the most rapid are tests which detect resistance to β-lactam antibiotics by the demonstration of β-lactamase in bacteria. This can be accomplished in a few minutes once a bacterial culture is available. However, since a wide range of β-lactamases are encountered to which various β-lactam agents display differential susceptibility, the tests are of limited value. Other methods employ turbidometric techniques to detect antibacterial activity by comparing the growth of bacteria exposed to antibiotic with a drug-free control over a time span which for fast growing organisms can be as little as 2 or 3 hours. Turbidometric results show discrepancies with more traditional methods with certain bacterium/drug combinations and there is controversy over the correct interpretation of these results. Equivalent methods are available for viruses, fungi and other parasites.

WO79/00834 describes a system for analysing chemical and biological samples using electrostatic fields.

Clearly, current approaches are less than ideal, and a rapid method for determining antibiotic susceptibility which can be widely applied and which provides results consistent with those obtained by conventional methods is highly desirable.

One of the first steps in drug discovery is to find one or more compounds that show a particular biological activity of interest. These first compounds, called leads, are usually discovered by screening, which means testing tens of thousands of random molecules for signs of activity.

Recent developments in automation have made it possible to screen tens of thousands of compounds per month, an effort that would have taken years previously. This has meant that there has been an exploding demand for “libraries” of random molecules to test Similar developments in automation and combinatorial chemistry have made it possible to synthesise thousands of distinct compounds per month, with a relatively small investment. These combinatorial libraries are made by forming all possible combinations of a series of sets of precursor molecules, and applying the same sequence of reactions to each combination. The distribution of precursors, and often the maintenance of reaction conditions, is carried out by a robotic synthesis system.

Lead molecules are typically only weakly active, and may be toxic or otherwise unsuitable for use as drugs. The next step in drug discovery is lead expansion, in which hundreds or thousands of variants of the most promising leads are made. These are tested, and some are typically found to be more active and/or less toxic than the original leads. This process can be iterative. The combinatorial library approach is also suitable for this lead expansion phase.

It is an object of the present invention to provide apparatus and methods which overcome some or all of these disadvantages.

### SUMMARY OF THE INVENTION

According to the present invention there is provided a method of identifying one or more micro-organisms in a fluid sample comprising the steps of:—

(i) optionally culturing the sample if necessary to increase the number of micro-organisms for a pre-determined range;

(ii) measuring the zeta potentials of any micro-organisms present;

(iii) optionally normalising the measurements taken in step (ii) such that they relate to standard conditions;

(iv) comparing said measured zeta potentials with a table of the zeta potentials of known micro-organisms to determine which, if any, of the known micro-organisms are present in the fluid.

By way of example there is provided a method of determining the presence of a specific micro-organism in a liquid solution which comprises applying an electric field across the solution, optically measuring the speed of movement of any micro-organism suspended in the solution as a result of the applied electric field, and identifying the presence of one or more specific organisms by comparing the measured values with those of known micro-organisms which have been measured under standard conditions.

The measured speed of movement or electrophoretic mobility is an indication of the electro-kinetic potential, also termed zeta potential, of the micro-organism and is dependent upon several physical properties of the micro-organism including size, shape, overall charge and surface charge distribution.

As well as permitting a solution to be analysed for the presence of a specific micro-organism, by comparing the zeta potentials measured during analysis of a solution with a table of the zeta potentials of known micro-organisms, the invention can enable rapid analysis of a solution to determine what mixture of micro-organisms are present.

According to a second aspect of the invention, there is provided an apparatus for determining the presence of a specific micro-organism in a liquid solution which comprises means for applying an electric field across a measurement cell containing the solution, a laser light source for illuminating the cell, detecting means for sensing light after impinging on a micro-organism suspended in the solution, means for analysing the scattered light to provide a measurement of the speed of movement of the micro-organism by which the laser light was scattered, and means for identifying the presence of the specific micro-organism upon measurement of a speed of movement matching that of the micro-organism.

The apparatus of the invention, as well as distinguishing between micro-organisms on the basis of their electo-kinetic potential, achieves improved sensitivity by allowing the scattering angle at which light is detected to be optimised for the micro-organism of interest as it has been found in practice that light from different micro-organisms is scattered in different directions.

It is preferred for the laser light source to comprise a laser, a beam splitter for dividing the light emitted from the light source into two coherent beams, means for directing the light beams along different directions and means for modulating the length of one of the light paths to create a variable interference pattern between the two beams at the cell. The effect of modulating the length of one of the light paths will be that the interference pattern will oscillate along the cell and light scattered by a stationary particle or micro-organism will pulsate at the frequency of the modulation. If the particle is moving however, it will move with the interference in one direction and in opposition to it in the opposite direction. Because of the Doppler effect, the frequency of modulation of the scattered light will differ from the frequency of modulation of the length of one of the light paths by an amount indicative of the rate of movement of the particle in the cell.

The detected frequencies of light scattered by moving particles when the applied electric field is reversed should be symmetrical about the modulation frequency of the light path. If, however, the electrodes of the cell are polarised then reversal of the potential applied to the electrodes will not result in an exact reversal of the applied electric field and the measurements obtained will not be symmetrical.

It is preferred to provide means for reversing the polarity of the electric field applied across the cell periodically to avoid polarisation of the electrodes as a result of an electrolytic reaction between the electrodes and ions in the solution.

The speed with which a given micro-organism will move through a solution when an electric field is applied will depend on certain factors other than the zeta potential, such as the pH and temperature of the solution. It is preferred to maintain these factors constant during measurement but it is alternatively possible to measure these parameters and compensate the measurement obtained.

According to a third aspect of the present invention there is provided a method of identifying the cause of an infection in a sample from human or animal body comprising the steps of:—

(a) culturing the sample if necessary to increase the number of micro-organisms to a pre-determined range;

(b) measuring the zeta potential of said micro-organisms using the method described herein;

(c) comparing the measured zeta potential(s) with values obtained on isolated micro-organisms.

This method can be used to direct a physician to an appropriate course of treatment. For example, not only can the method flag up the infecting species, but it can also list a range of possible drug treatments known to be effective against the micro-organisms which have been identified. The physician would then select the appropriate treatment based on this information, the symptoms exhibited by the patient and the physicians skill and experience.

According to a further aspect of the invention there is provided a method for antibiotic sensitivity testing.

In one embodiment, the method comprises:

(a) measuring the zeta potential of one or more micro-organism in a sample,

(b) adding to said sample an antimicrobial agent,

(c) measuring again the zeta potential of the said one or more micro-organisms in said sample,

(d) determining the change in zeta potential between said measurements and correlating said change to either:

(i) the antimicrobial sensitivity of said one or more micro-organisms in said sample, or

(ii) the antimicrobial activity of said antimicrobial agent.

In a further embodiment, the step correlating said change in zeta potential to either the antimicrobial sensitivity of a micro-organism or to the antimicrobial activity of a an antimicrobial agent may be automated.

The present invention enables the following objectives and advantages to be accomplished.

It is an object of the present invention to provide a method for rapidly assessing the susceptibility of a patient or veterinary sample to a spectrum of antimicrobial agents in order to determine the most appropriate treatment to be initiated. Advantages of the present invention include avoidance of the development of drug-resistant strains; economic dispensing of medicines; and an increase in the quality of patient care.

Another object of the present invention is to provide a method for assessing emerging drugs for antimicrobial activity. Advantages of the present invention include determination of MIC values; investigation of the effects of the candidate drugs on microbial physiology; and determination of the mechanism of action of the candidate drug.

A further object of the present invention is to provide a method for screening for agents having antimicrobial activity. Advantages of the present invention include the ability quickly to perform large-scale, automated screening; to identify drugs which affect cell vitality but not cell viability.

A yet further object of the present invention is to provide an automated method for screening agents for antimicrobial activity.

### DESCRIPTION OF THE DRAWINGS

The invention will be further described, by way of an example only, with reference to the accompanying drawings wherein:—

FIG. 1 illustrates a stylised diagram of a yeast cell;

FIG. 2 illustrates graphically the diffuse double layer around a sphere of moderate potential;

FIG. 3 illustrates schematically a typical double beam apparatus for measuring zeta potentials;

FIG. 4 shows the Zeta potential profile of a control sample of S pneumoniae;

FIG. 5 shows the zeta potential profile of a sample of S pneumoniae treated with penicillin.

### DESCRIPTION OF THE PREFERRED EMBODIMENTS

The present aspects and embodiments represent currently the best ways known to the applicant of putting the invention into practice. But they are not the only ways in which this could be achieved. They are illustrated, and they will now be described, by way of example only.

Techniques and apparatus for measuring electrophoretic mobility and zeta potential are known per se and have been used in the analysis of certain colloidal suspensions, such as paints. The present invention is, however, based on the discovery that the zeta potential of a micro-organism is a parameter which under properly controlled conditions, can act as a reliable indicator of the presence of that specific micro-organism.

Microelectrophoresis is a division of the science of electrophoresis and deals specifically with particles in the region of less than 20 μm and with a minimum dimension in the order of 0.01 μm. Below this limit, objects are considered to be of molecular dimensions. There is considerable blurring of this lower border especially when dealing with macromolecules. Electrophoresis has long been the method of choice for identifying many charged molecular species, such as proteins, nucleic acid and amino acids. One much publicised type of electrophoresis is DNA finger printing which allows the separation and matching of two samples of DNA.

Micro-organisms of all types, from bacteria to viruses fall into the size range applicable to microelectrophoresis. Viruses are particularly interesting because they are composed mostly of protein and nucleic acid and tend to have a relatively stable, uniform structure. As such, they provide a direct link with conventional electrophoresis techniques.

When any surface is placed in contact with an aqueous solution the surface acquires a surface charge which is usually negative. The origins of this are either absorption of ions from solution or by the ionisation of charged groups on the surface. In the case of micro-organisms both of these mechanisms can be seen to operate and the observed surface charges are usually negative, which is the commonly observed sign. The use of this charge to identify specific micro-organisms is what makes this technology both novel and powerful. Importantly, the method is not size dependent so both large and small micro-organisms can be identified.

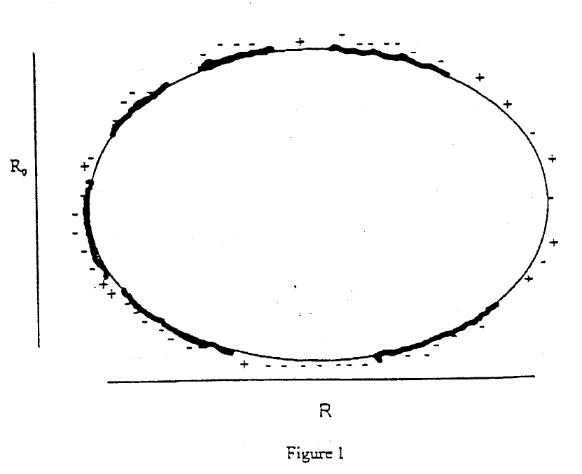

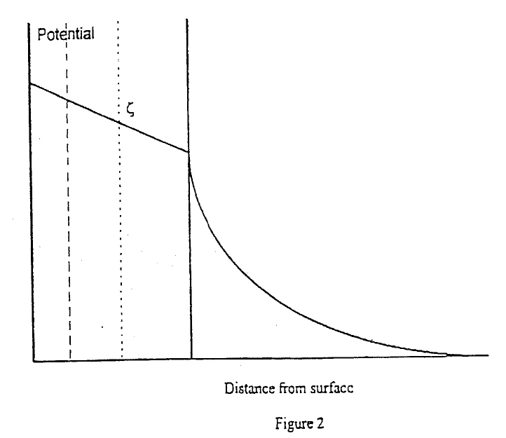

For purposes of microelectrophoresis FIG. 1 describes how the micro-organisms can be regarded from this view point. The shaded areas represent areas of hydrophobic surface at which charge builds up at the surface through contact absorption. Also shown at this surface are the charges native to the surface of the micro-organism and specifically absorbed at the surface. The charge produces an electrical double layer and the electrical potential varies as described by FIG. 2. FIG. 2 shows the distribution of charge across a double layer at the surface of a hypothetical micro-organism. The charge at the shear surface is the zeta potential; beyond this the charge decays away rapidly in the bulk solution.

The surface charge of an idealised micro-organism arises from two sources. First groups on the surface of the micro-organism, such as carboxylate groups, may be ionised. Secondly, ions may be adsorbed from solution onto the surface of the micro-organism, particularly at hydrophobic regions on the surface where anionic groups may be preferentially adsorbed.

Apparatus for measuring zeta potentials are known per se. One such example is the ZETASIZER 2000 (TM) made by Malvern Instruments.

This instrument, as it name suggests, is used to measure particle size. It utilises a dual laser beam and a photodetector is arranged to measure deflected light at a fixed scattering angle.

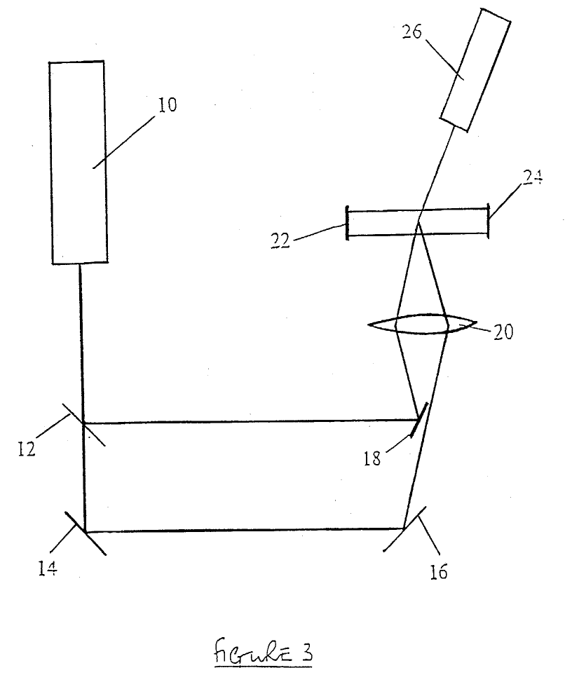

A typical double beam instrument is shown in FIG. 3 which illustrates a neon-helium laser 10 producing a red laser beam. The beam is incident upon a beam splitter 12 that allows half the incident light energy to pass through it towards a mirror and modulator 1 and reflects the other half towards a mirror 18. The mirror and modulator 14 is a mirror vibrated by a piezoelectric vibrator that reflects the incident beam towards a further stationary mirror 16. The two halves of the split laser beam are focused by a lens 20 an a cell 22 that contains a liquid to be analysed. An electric field that is reversed periodically is applied across the cell by means of electrodes 22 and 24 and this causes micro-organisms in the liquid to move with a characteristic velocity towards the electrode that instantaneously has the opposite polarity to the net charge on the micro-organism. When the micro-organism is illuminated by the laser light it scatters the incident light in a predetermined direction and this light is sensed by means of a detector 26.

The two halves of the split laser beam travel different distances before reaching the cell 20. In the case of the half that is reflected by the mirror and modulator 14, the path length is modulated periodically. When they are recombined, the two halves of the split laser interfere with one another and the interference pattern will move across the cell. As a result, a stationary particle suspended in the cell will scatter light at a frequency related to the frequency of modulation of the mirror 14. If, however, a particle is moving towards one end of the cell 20 or the other, then while it is moving with the interference pattern, light scattered from it will have a lower frequency and conversely when the particle is moving in the opposite direction to the interference pattern light scattered from it will have a higher frequency. By analysing the frequency of the scattered light sensed by the detector 26 it is possible to determine the speed and direction of movement of the particle.

The speed of movement is related to the distribution of charge about the surface of the particle and is indicative of the so-called zeta potential. Depending on the shape of the particle, it will align itself differently in the applied electric field and for any specific micro-organism there will be an angle at which the intensity of the scattered light is a maximum. The apparatus may therefore be used to determine the presence of a specific micro-organism in the solution being analysed by setting the detector 26 to the scattering angle associated with the micro-organism and monitoring the output of the detector for the frequencies corresponding to the previously determined speed of movement of the micro-organism in the applied field.

It is preferred to take steps to prevent polarisation of the electrodes 22, 24 as such polarisation may affect the electric field as sensed by particles suspended in the liquid in the cell 20. Polarisation may be prevented by periodically switching the polarity of the electric field. Such switching will in itself prevent polarisation but additionally it will allow polarisation to be detected as the detected frequencies will not be symmetrical about the frequency of movement of the bands of the interference pattern.

The detector type used in this application can take a number of forms. However, we have found that commercially available solid state photodiodes such as Radio Spares Model AEPX 65 (Stock No 8461749) work well.

The nature of the data captured takes the form of a series of exponential decays. These represent the length of time taken for the scattered light to decay to zero, this is effectively the particle velocity. The exponential decays are converted to mean normal distributions. These are collected and transformed graphically by the method of Eishus to give mobilities and zeta potentials as follows:—

$${g}^{( 1 )} ( t ) = \frac{2}{t} sinh {( Δ Γ )}_{2}^{t} {Σ}_{j - 1}^{m} {G}_{i} exp ( - t Γ ) .$$

t=time

g⁽¹⁾(t)=nonmalised electric field autocorrelation function

G₁=normalisation constants and are obtained from fitting experimental values of Γ to the summation part of the above equation.

Values of j are usually limited to 3 and are identified with the mean, standard deviation and skewness of the data.

For the homodyne case the g⁽¹⁾(t) is obtained from:

g⁽²⁾(t)=1+[g⁽¹⁾(t)]² where g⁽²⁾(t) is obtained from

g⁽²⁾(t)=1+e⁻²^{_{Γ}}^{t} and is known as the homodyne autocorreiation coefficient

(Ref: D. E. KOPPEL J Chem Phys 1972 57 4814)

This method of handling the data measured is contained in a software programme as a means for analysing the scattered light. In addition, the software can contain a library or table of recorded measurements for known micro-organisms. A comparator programme can be run such that the operator receives as a print-out or screen message a list of those micro-organisms considered to be present.

In this context the term look-up table is used as a generic term to encompass any compilation of zeta potential measurements obtained for known micro-organisms. This look-up table can take a variety of forms. It may, for example, be a physical table whereby comparisons are made manually. Alternatively, and preferably, the “table” can be a computer database of results. A comparator programme then compares a measured value, or series of measured values, against known results. Limits may be included within the programme such that a “best fit” only answer is provided or a range of “possibilities” can be given. The comparator programme may also advantageously contain the necessary calculations to normalise the measured zeta potential values such that they relate to standard conditions.

An apparatus of the general type described above has been used to measure the electrophoretic mobility and zeta potentials of known micro-organisms. The machine was calibrated using a standard NaCl solution having a conductivity of 0.15 mS and a pH of approximately, 7.0. Test samples were normalised to this conductivity by use of the Briggs equation, which in this case takes the form:

Kₛₒₗ=(K_{obs}+Kₛₜ)/Kₛₜ

Where the K's are solution, observed and standard respectively.

Most encouragingly, different samples of the same bacteria gave constant results. Unexpectedly, the zeta potential peaks for different organisms occurred at different and distinguishable voltages. The sensitivity of the present system is such that different organisms can be identified uniquely, even from a mixture of organisms. Typical results are shown in Table 1. This work shows how micro-organisms can be identified one from another in dilute aqueous solution at pH 7. These experiments were carried out by injecting a solution containing in the region of 100,000 micro-organisms per millilitre into an apparatus as shown in FIG. 3 or an apparatus of the type described above.

TABLE I

| | Mean/Zeta Potential | |
|---|---|---|
| Micro-organism | (mV) | Standard Deviation |
| Campylobactor jejuni | −7.5 | 0.945 |
| Listeria monocytogenes | −11.7 | 1.08 |
| Salmonella (Wild) | −9.76 | 1.00 |
| E. Coli (0157) | −18.6 | 2.15 |
| Shigella sonnei | −27.2 | 1.1 |
| Listeria innocula | −11.9 | 0.81 |
| Bacillus cereus | −18.8 | N/A |
| Staphylococcus aureus | −17.8 | N/A |
| Proteus mirabilis | −21.5 | N/A |
| Entrococcus faecalis | −15.3 | N/A |
| Saccharomyces cerevisiae | −23.4 | 0.5 |
| S Carlburgensis | −27.3 | 0.6 |
| Saccharomyces cerevisiae | −3.0 | 0.4 |
| (non-viable) | | |
| S Carlburgensis | −2.0 | 0.2 |
| (non-viable) | | |

It is assumed that the data is normally distributed around the mean, which has been established for yeasts in normal growth. This allows calculation of the goodness of separation which in this case it is represented by the chi squared analysis shown in Table II.

TABLE II
Chi squared analysis of date in Table I

| Micro- | | | | E. Coli | Shi- |
|---|---|---|---|---|---|
| organism | Campylobactor | Listeria | Salmonella | (0157) | gella |
| Campylobactor | | | | | |
| Listeria | .024 | | | | |
| Monocytogenes | | | | | |
| Salmonella | 0.21 | 0.355 | | | |
| E. Coli (0157) | 0 | 0.019 | 0.001 | | |
| Shigella | 0 | 0 | 0 | 0.015 | |
| Listeria | 0.013 | 0.949 | 0.49 | 0.002 | 0 |
| Innocula | | | | | |

Table II indicates that the only two members of the table that are not separable are the two members of the listeria group of organisms.

The observation that two different strains of the Listeria bacteria cannot be differentiated one from another does not greatly detract from this method. The information that a patient is suffering from Listeriosis, as opposed to an E Coli or other bacterial infection, is of great assistance to a medical practitioner. A course of treatment can be started immediately which will combat all Listeria strains which are commonly found in the human population.

However, it is possible to further enhance the sensitivity of this method by incorporating one or more of the additional steps, which make the identification process more discriminatory, or provide confirmation of micro-organism identity:

1. Culturing the sample in a medium selective for a particular micro-organism or class of micro-organisms, whereby the micro-organism or micro-organisms of interest grows in preference to other micro-organisms that may be present. A wide range of selective media is available from Oxoid Ltd and is described in their catalogue. This provides a method of distinguishing between micro-organisms that otherwise have very similar zeta potentials.

2. Contacting the sample with an agent known to kill one or more of the micro-organisms present, and measuring the zeta potential a second time. The elimination of the zeta potential corresponding to the micro-organism of interest is a confirmatory test if the agent is specific for the micro-organism of interest. The agent may be an antibiotic, antibacterial or other selective poison. The same end may also be accomplished by contacting the sample with an agent that specifically causes the agglomeration of the micro-organism, such as an antibody or other immunological reagent, whereby the micro-organism is precipitated out of solution.

3. Measuring the zeta potential at a second pH and comparing said zeta potential with a second table of the zeta potentials of known micro-organisms at this second pH. The diversity of the ionising groups on the surface of the micro-organism, which contribute to the zeta potential, means that a distinction can be made between two micro-organisms which have a similar zeta potential at one pH, but not at another.

The facility to perform zeta potential measurements at different pH's is a key aspect of one of the embodiments of the invention. It provides a means of distinguishing between two micro-organisms which would otherwise be difficult or impossible to separate.

In one embodiment this process can be automated. Having taken a zeta potential measurement at a first pH, an aliquot of acid or base can be introduced into the measurement cell and the reading repeated. In fact, a series of pH's can be used by the incremental additions of acid or base.

Dosage means to infect a solution of acid or alkali is known per se to the person skilled in the art. Typical bases which can be used are sodium or potassium hydroxide or an amine base such as triethylamine. Acids include hydrochloric or sulphuric acid or organic acids such as acetic or citric acid.

The apparatus may advantageously include a pH meter such that the pH of the solution in the cell is measured and recorded automatically.

The uniformity of virus structures would give every indication that viruses can be distinguished one from another in a similar way.

As such, this method represents an important new method of diagnosis which can be performed in vitro on a sample removed from the human body. The method does not have to be performed by a medical practitioner but can be readily carried out by a technician. The technician need not have any medical knowledge in order to carry out this method effectively and requires only the most basic of training.

The method provides the medic with a clear indication of which pathogenic bacteria, or combination of bacteria, are present in the patient's sample. From that information and the symptoms experienced by the patient, the medic can then formulate the most appropriate treatment. It provides, in effect, an intermediate result upon which to decide on a suitable course of treatment.

Except as stated above, it is preferred that the pH value of the solution and its temperature should always be the same during measurement as the pH may affect the measured zeta potential but it is alternatively possible to compensate the obtained measurement for changes in pH and temperature.

However, an important advantage of the present invention is that while the chemical composition of the micro-organism is the same whether it is alive or dead, the zeta potential changes when the micro-organism dies because the charge distributed around the micro-organism is affected by the chemical processes taking place while the organism is alive, especially near its outer periphery. If, therefore, steps have been taken to eliminate a micro-organism that is the cause of an infection, the present invention can be used to determine if such steps have been effective.

The invention has been described in relation to identifying organisms in a solution. In this context the term “solution” has a very broad meaning. It refers to any liquid, organic or inorganic, which may contain a micro-organism. It includes, by way of example only, notionally pure water, brewing worts, body fluids such as urine and diluted samples of these, plant extracts or swabbings and solutions obtained by bubbling a gas through a liquid in order to entrain any micro-organisms present in the gas. In this way contaminations in gaseous fluids may also be investigated using the present method.

This method is not limited to the foregoing micro-organisms in Table I. Potentially any pathogenic micro-organism or class of micro-organism can be identified using this method and the preferred treatment(s) selected.

Where very small numbers of bacteria are present in the initial sample then it may be necessary to culture the micro-organism using methods well-known in the art.

The bacterial count in the urine from patients having urinary infections is in the order of 10⁶ to 10⁷ cells per ml. These levels are easily and rapidly detected using the method of the present invention, and it is anticipated that an indication of the infecting pathogen will be provided within 5 minutes.

Food and water samples however tend to contain rather fewer bacteria per ml, and for these the sample will need to be cultured. The sample may be mixed with, for example, McConkey's broth. After a predetermined time, when the number of cells present per ml will have increased to 10⁵ or more, the cultured sample may be analysed for the presence or absence of micro-organisms using the method of the present invention. No filtration is necessary and the broth ingredients do not interfere with the analysis.

Food samples are prepared, for example, by swabbing the surface of the food or macerating the food. Thus Salmonella could be detected in a chicken neck flap swab within 7 h, and indications are that 200 cells per ml of Salmonella (a level which is considered to be infective) could be detected within 2.5 h culture time.

These timescales contrast markedly with the methods currently available where results take days not hours to come through. Recent food poisoning and meningitis scares have shown that patients can deteriorate very substantially or even die over this time period. Providing there are sufficient micro-organisms in the sample then a result can be obtained in minutes using the present method. Importantly, the culture media used to multiply micro-organisms do not interfere so no filtration or only crude filtration is required.

This method, and particularly the method of data processing is applicable to a wide variety of samples including human and animal body fluids, plant extracts, food, drink and notionally pure water.

In a further aspect of the invention, this method can be applied to provide a method of antibiotic sensitivity testing. The method comprises measuring the zeta potential of a microbial sample, which may be a known micro-organism or a clinical or veterinary sample; the sample is then exposed to an antimicrobial agent; subsequent measurement of the zeta potential indicates both the potency of the antibacterial material, and the sensitivity of the microbial sample to that material.

In another embodiment, the approach may be applied to assessing the antimicrobial activity of a material. Without wishing to limit the scope of the invention, the material may be a compound or mixture of compounds isolated from a natural source, it may be a compound that is a derivative of a known family of antimicrobial agents, or it may be one or more compounds from a library of compounds produced by combinatorial chemistry techniques. Typically these libraries contain between 5 and 50 variants of a candidate therapeutic agent.

In a further embodiment, the approach may be applied to determining the antimicrobial susceptibility of a clinical or veterinary sample obtained from a patient or animal suspected of suffering from a microbial infection to determine the most effective treatment of the infection.

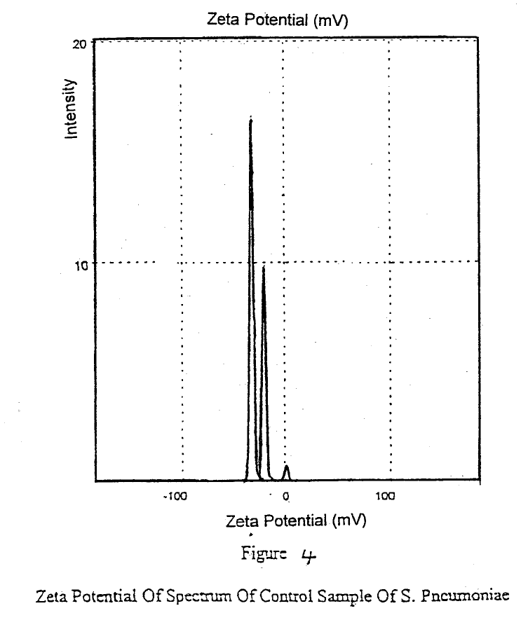

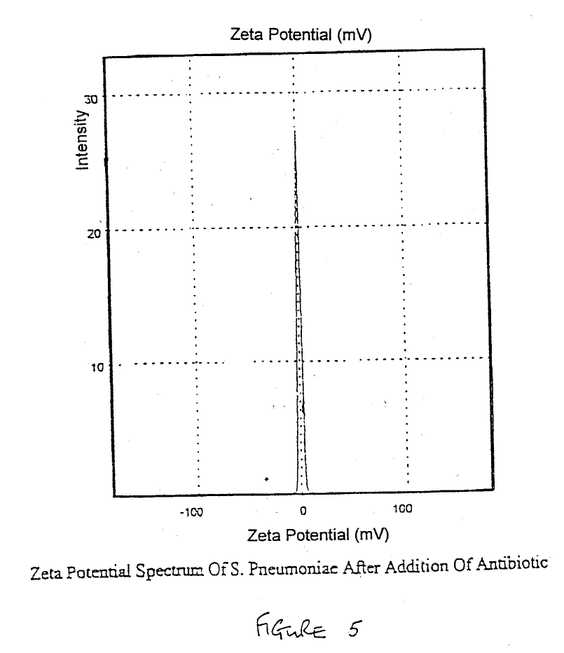

The following Example illustrates a particular embodiment of the invention: Streptococcus pneumoniae was grown for about 4 h in laked horse blood medium. Aliqouts, representing about 10⁵ cells, were transferred to two tubes containing growth medium: one of these also contained penicillin at 0.01 g/ml. The zeta potential of both samples was measured using a ZETASIZER 2000 (RTM) from Malvern Instruments. The tubes were incubated at 37° C. for 3 h, and the zeta potential of each measured again. FIG. 4 shows the zeta potential profile of the tube containing the growth medium and S pneumoniae only; FIG. 5 shows the zeta potential of the tube additionally containing penicillin. There is a clear difference in these profiles. In both Figures, the peak at 0 mV represents dead cells. The zeta potential of all dead micro-organisms is close to 0 mV: without wishing to be bound by a particular theory, it is believed that these changes are brought about by the vacuolation of the cells, resulting in the zeta potential decaying to about 0 mv. Living, healthy cells of S pneumoniae have a zeta potential of about −27.9 mV, as shown in FIG. 4 and Table III. The other peak in FIG. 4 is believed also to be cells of S pneumoniae, but which are non-viable.

TABLE III
Mean Zeta Potentials for S pneumoniae

| Sample | Mean Zeta Potential (mV) |
|---|---|
| Antibiotic Present | −0.3 |
| No Antibiotic Present | −27.9 |

Thus the present invention is directed toward a method for assessing antimicrobial sensitivity.

In one aspect, the invention provides a method for rapidly assessing the susceptibility of a patient or veterinary sample to a spectrum of antimicrobial agents in order to determine the most appropriate treatment to be initiated. This process could be automated, so that a patient would have a sample taken during the morning by a nurse or technician, the sample would be analysed according to the present invention, and a prescription for the most appropriate antibiotic would be available later that day.

A machine is envisaged in which a sample from a patient is divided into x samples and each sample is incubated to provide about 10⁵ cells per sample. Each sample is then treated with a different antibiotic and incubation continued. After set periods of time the zeta potential of each sample is measured.

The antibiotic associated with the sample whose zeta potential tends to zero quickest is the treatment of choice. A printer associated with the machine can then print out a prescription for checking and signature by the medical practitioner.

This method is not a method of diagnosis as such. It simply provides the medical practitioner with information on which agent(s) kills the micro-organism(s) most satisfactorily in vitro. It can be carried out by a technician with no medical knowledge and only basic training.

In another aspect the invention may be applied to assessing emerging drugs for antimicrobial activity.

In a further aspect the invention can be applied to screening for agents having antimicrobial activity.

The foregoing describes how the approach may be used for antibacterial agents: the same approach may be applied to antifungal, antiviral and anti-parasitic agents.

1. A method of identifying one or more micro-organisms in a fluid sample comprising the steps of:—
(i) optionally culturing the sample if necessary to increase the number of micro-organisms for a pre-determined range;
(ii) measuring the zeta potentials of any micro-organisms present;
(iii) optionally normalising the measurements taken in step (ii) such that they relate to standard conditions;
(iv) comparing said measured zeta potentials with a table of the zeta potentials of known micro-organisms to determine which, if any, of the known micro-organisms are present in the fluid.

2. A method according to claim 1 wherein a first zeta potential measurement is obtained on a fluid sample at a first pH and a second zeta potential measurement is obtained at a second pH, the difference, if any, between the first and second measurements being used to distinguish between micro-organisms with otherwise similar zeta potentials.

3. A method according to claim 1 or claim 2 wherein in optional step (i) the sample is cultured in a medium selective for a particular organism or class of organisms, said modification providing a method of distinguishing between micro-organisms that otherwise have similar zeta potentials.

4. A method according to any preceding claim wherein in a further step the sample is contacted with an agent known to kill one or more particular micro-organism(s) and measuring the zeta potential a second time, the elimination of the zeta potential corresponding to a micro-organism of interest providing confirmation of its presence.

5. A method according to any preceding claim wherein said sample is a sample taken from a human or animal body, said method providing an indication of the cause of an infection in said human or animal body.

6. A method according to any preceding claim wherein the comparison between the measured zeta potential(s) and said table of known zeta potentials is carried out using a computer programme, the result being displayed on a computer screen or by way of a printout.

7. A method of identifying one or micro-organisms in a fluid substantially as herein described with reference to and as illustrated in any combination of the accompanying drawings.

8. A method of identifying the cause of an infection in a sample from human or animal body comprising the steps of:—
(a) optionally culturing the sample if necessary to increase the number of micro-organisms to a pre-determined range;
(b) measuring the zeta potential of any micro-organisms present;
(c) comparing said measured zeta potentials with a table of the zeta potentials of known micro-organisms to determine which, if any, of the known micro-organisms are present in the fluid.

9. A method according to claim 8 wherein a first zeta potential measurement is obtained on a fluid sample at a first pH and a second zeta potential measurement is obtained at a second pH, the difference, if any, between the first and second measurements being used to distinguish between micro-organisms with otherwise similar zeta potentials.

10. A method according to claim 8 or claim 9 wherein in optional step (a) the sample is cultured in a medium selective for a particular organism or class of organisms, said modification providing a method of distinguishing between micro-organisms that otherwise have similar zeta potentials.

11. A method according to any of claims 8 to 10 wherein in a further step the sample is contacted with an agent known to kill one or more particular micro-organism(s) and measuring the zeta potential a second time, the elimination of the zeta potential corresponding to a micro-organism of interest providing confirmation of its presence.

12. A method according to any of claims 8 to 11 wherein the comparison between the measured zeta potential(s) and said table of known zeta potentials is carried out using a computer programme, the result being displayed on a computer screen or by way of a printout.

13. A method of identifying the cause of an infection on a human or animal body substantially as herein described with reference to and as illustrated in any combination of the accompanying drawings.

14. A method of antibiotic sensitivity testing comprising:—
(a) measuring the zeta potential of one or more micro-organism in a sample,
(b) adding to said sample an antimicrobial agent,
(c) measuring again the zeta potential of the said one or more micro-organisms in said sample,
(d) determining the change in zeta potential between said measurements and correlating said change to either:
(i) the antimicrobial sensitivity of said one or more micro-organisms in said sample, or
(ii) the antimicrobial activity of said antimicrobial agent.

15. A method of antibiotic sensitivity testing substantially as herein described and with reference to any combination of the accompanying drawings.

16. An apparatus for carrying out the methods described in claims 1 to 15 inclusive comprising:—
(i) means for applying an electric field across a measurement cell;
(ii) a light source for illuminating the measurement cell;
(iii) detecting means for detecting light scattered by micro-organisms present in the cell;
(iv) means for analysing the scattered light to provide a measurement of the speed of movement of a micro-organism(s);
(v) means for computing the zeta potential of said micro-organism(s);
(vi) means for comparing said measured zeta potentials with the zeta potentials of known organisms;
characterised in that the apparatus further comprises a means for normalising the zeta potential measurements to facilitate comparison with results for known micro-organisms measured under standard conditions.

17. An apparatus as claimed in claim 16 wherein the means for comparing measured and reference zeta potentials comprises a comparator computer programme in which the measured value is compared with values in one or more look up tables, the results being displayed on a VDU screen or printed out on a printer.

18. An apparatus substantially as herein described with reference to and as illustrated in any combination of the accompanying drawings.
